(19) United States
(12) Patent Application Publication
(10) Pub. No.: US 2005/0250658 A1
(43) Pub. Date: Nov. 10, 2005

(54) Methods of conditioning the skin and articles of commerce

(76) Inventors: Christopher Dean Putman, West Chester, OH (US); Rebecca Ann Taylor, Cincinnati, OH (US); June Turkansis Brennock, Cincinnati, OH (US); Mannie Lee Clapp, Mason, OH (US); Roberto Escobosa, Loveland, OH (US); Yamilca Del Carmen Thoms, Loveland, OH (US); Chow-Chi Huang, West Chester, OH (US); Laura Gates Rockstroh, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/123,408

(22) Filed: May 6, 2005

Related U.S. Application Data
(60) Provisional application No. 60/569,086, filed on May 7, 2004.
(60) Provisional application No. 60/626,133, filed on Nov. 8, 2004.

(51) Int. Cl. A61K 7/50

(52) U.S. Cl. 510/130

(57) ABSTRACT

An article of commerce is provided which includes (a) an inverted container that includes a valve and an aperture; (b) a personal care composition contained in the inverted container, and (c) a hanging feature. An article of commerce is provided that includes (a) an inverted container comprising a valve; (b) an in-shower moisturization composition contained in said inverted container; and (c) a set of instructions in association with the inverted container, the set of instructions comprising instructions to dispense the in-shower moisturization composition from the inverted container, contact skin with the in-shower moisturization composition during showering or bathing, rinse the skin, and dry the skin. A personal care kit is provided and methods for utilizing the personal care compositions are provided which enhance skin and/or hair moisturization and conditioning.

### CROSS REFERENCE TO RELATED APPLICATIONS

This application claims the benefit of Provisional Application Nos. 60/569,086, filed May 7, 2004, and 60/626,133, filed Nov. 8, 2004.

### TECHNICAL FIELD

The present invention relates to an article of commerce comprising an inverted container, comprising a valve and an aperture, containing a personal care composition.

### BACKGROUND OF THE INVENTION

Personal care compositions are well known and widely used for cleansing and moisturizing skin and hair, delivering actives, hiding imperfections and to reducing the oiliness/shine. The efficacy of these types of compositions is directly related to their frequency of use. An early invention which helped to enhance the efficacy of these products through an increase in ease of use and hence compliance was the utilization of a pump type system to dispense the product out of a container. This provides an ease of use of the conditioning composition and promotes dispensing the composition multiple times during a single conditioning application event to designated areas of the body or to the entire body if the user desires.

Unfortunately, the use of a pump system in the bathroom or shower to deliver a conditioning composition has drawbacks. The placement of a container having a pump as the means of dispensing the product in the shower or bath requires some type of a shelf or ledge which allows the user to dispense the product out of the container. With the space in the shower or bath being limited, a typical shower or bath does not have a location, like a shelf or ledge, to place a container having a pump. Additionally, due to the difficulty in using the pump system in a shower or bath, the user is discouraged from dispensing the composition multiple times during a single conditioning application.

There are currently marketed products which comprise personal care compositions in an inverted container. These products can be taken into the shower or bath and can be used to cleanse condition and moisturize the skin and/or hair. However, these personal care composition compositions are placed in an inverted container that must be picked up and put down each time the user dispenses the product and the container must be opened and closed, if the user wants to dispense the product multiple times. Additionally, when the inverted containers are in an open position, the compositions tend to leak out of the container. The efficacy of these types of inverted containers in providing conditioning benefits using conditioning compositions is greatly reduced due to the user's inability to dispense multiple doses of composition without the need to open and close the inverted container multiple times, thereby discouraging multiple doses being used.

Thus, a need still exists for a method of using an inverted container comprising a personal care composition that the user can dispense the product multiple times without the need to open and close the container and pick up and put down the inverted container.

The efficacy of a personal care composition in the shower or bath is enhanced when the composition is packaged in an inverted container of the present invention which comprises a valve. Additionally, the incorporation of the valve located in an orifice of a cap portion of the inverted container encourages and allows the user to dispense the product multiple times from the container without the need to continually open and close an orifice cover results in enhanced cleansing, moisturization and conditioning of the user's skin and/or hair. Moreover, the incorporation of the aperture and the hanging feature allows the user to dispense the product multiple times without taking up valuable space in the shower or bath.

### SUMMARY OF THE INVENTION

The present invention relates to an article of commerce that comprises (a) an inverted container comprising a valve and an aperture; (b) a personal care composition contained in said inverted container, and (c) a hanging feature. The present invention also comprises a personal care kit that comprises (a) an article of commerce comprising an inverted container comprising a valve and an aperture and a personal care composition contained in the inverted container; and (b) a hanging feature. The present invention further comprises an article of commerce that comprises (a) an inverted container comprising a valve, (b) an in-shower moisturization composition contained in said inverted container; and (c) a set of instructions in association with the inverted container, the set of instructions comprising instructions to dispense the in-shower moisturization composition from said inverted container, contact skin with the in-shower moisturization composition during showering or bathing, rinse the skin, and dry said skin.

In one embodiment of the present invention the invention relates to a method of moisturizing skin during showering or bathing, the method comprising the steps of: (a) dispensing a personal care composition from an inverted container comprising a valve; (b) contacting the skin with the personal care composition (c) rinsing the skin with water, d) drying the skin. The personal care composition is preferably applied to the entire body.

### BRIEF DESCRIPTION OF THE DRAWINGS

FIG. 1 is a perspective view of an inverted container of the present invention.

FIG. 2 is a perspective view of a valve of the present invention.

FIG. 3 is a perspective view of an inverted container comprising a slit valve of the present invention with a removable orifice cover.

FIG. 4 is a perspective view of an inverted container comprising a slit valve of the present invention with a swing orifice cover.

FIG. 5 is a perspective view of the slit valve and swing orifice cover of FIG. 5.

FIG. 6 is a perspective view of an inverted container of the present invention with a hanging feature and suction cup.

FIG. 7 is a front view of an inverted container of the present invention.

FIG. 8a is a back view of an inverted container of the present invention.

FIG. 8b is a perspective view of a portion of the inverted container shown in FIG. 8a.

FIG. 9 is a side view of the inverted container of the present invention.

FIG. 10 is a perspective view of the hanging feature of the present invention.

### DETAILED DESCRIPTION OF THE INVENTION

All percentages and ratios used herein are by weight of the total composition and all measurements made are at 25° C., unless otherwise designated.

“Benefit agents” useful herein may be categorized or described herein by their cosmetic and/or therapeutic benefit or their postulated mode of action. However, it is to be understood that the active and other ingredients useful herein can in some instances provide more than one cosmetic and/or therapeutic benefit or operate via more than one mode of action. Therefore, classifications herein are made for the sake of convenience and are not intended to limit an ingredient to the particularly stated application or applications listed.

The term “dermatologically-acceptable,” as used herein, means that the compositions or components thereof so described are suitable for use in contact with human skin without undue toxicity, incompatibility, instability, allergic response, and the like.

The term, “personal care composition” as used herein refers to unless otherwise specified, refers to the compositions of the present invention, wherein the compositions are intended to include compositions for topical application to the skin or hair.

The term “safe and effective amount” as used herein means an amount of a compound, component, or composition sufficient to significantly induce a positive benefit, preferably a positive skin appearance or feel benefit, including independently the benefits disclosed herein, but low enough to avoid serious side effects, i.e., to provide a reasonable benefit to risk ratio, within the scope of sound medical judgment.

The term “topical application”, as used herein, means to apply or spread the compositions of the present invention onto the surface of the skin.

A “valve” as used herein means a any of numerous mechanical devices by which the flow of a personal care composition may be started, stopped, or regulated by a movable part that opens, shuts, or partially obstructs one or more ports, orifices or passageways. Valves as used herein may be any valve known in the art including but not limited to a manually openable valve; a pressure activated valve; and a slit valve. A “pressure activated valve” as used herein means a valve having a closed position as to prevent unwanted leakage of the composition when the inverted container is not in use. When the inverted container is in use, the valve has an open position for dispensing the composition through an orifice in response to manual squeezing forces being applied to the inverted container. The valve returns to its closed position upon removal of the manual squeezing forces which results in stopping the dispensing of the composition through the orifice. A “slit valve” as used herein means a valve having incision(s) that have a closed position as to prevent unwanted leakage of the composition when the inverted container is not in use. When the inverted container is in use, the incision(s) of the “slit valve” has an open position for dispensing the composition through an orifice in response to manual squeezing forces being applied to the inverted container. The “slit valve” returns to its closed position upon removal of the manual squeezing forces which results in stopping the dispensing of the composition through the orifice.

Article of Commerce

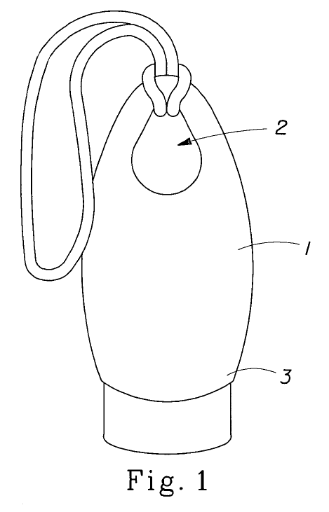

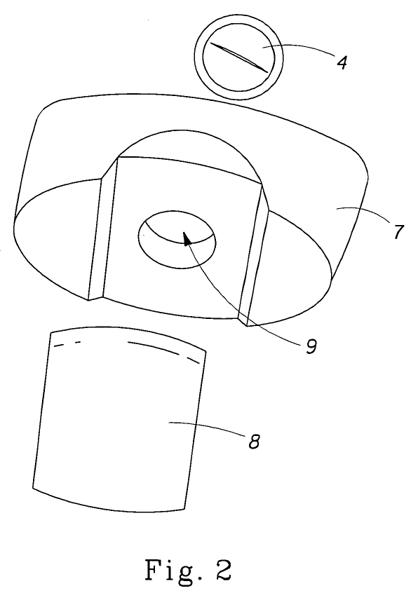

The present invention encompasses articles of commerce illustrated in FIG. 1-10. The article of commerce comprises an inverted container 1 comprising a valve 4 shown in FIG. 2-6, as well as a personal care composition contained in the inverted container 1. The present invention also comprises an article of commerce that comprises (a) an inverted container 1 comprising a valve 4, (b) an in-shower moisturization composition contained in the inverted container 1; and (c) a set of instructions in association with the inverted container 1, the set of instructions comprising instructions to dispense the in-shower moisturization composition from the inverted container 1, contact skin with the in-shower moisturization composition during showering or bathing, rinse said skin, and dry said skin.

The inverted container 1 may include tubes, bottles, tottles or pouches. The aperture 2 may be formed in the container at the time of manufacturing or could be formed in a subsequent operation such as applying an adhesive tab that has been die-cut to include an aperture. The preferred inverted container 1 is a blow molded plastic container whereby the aperture 2 is formed integrally into the container 1 at the time of manufacture. The inverted container 1 may include multiple layers. The inverted container 1 may be made from any suitable polymeric material including but not limited to polyethylene, polyurethane, polyester, nylon or polypropylene. The preferred material for the bottle is polyethylene. The inverted container 1 may be comprised of transparent, translucent, or opaque materials. The inverted container 1 may be comprised of a combination of materials with these properties.

FIG. 1 illustrates one possible embodiment of an inverted container 1 comprising an aperture 2 adjacent the top portion 3 of the inverted container 1. This aperture 2 is non-dispensing and allows the user to hang the inverted container 1 from a support in the shower or bath. As well, the aperture 2 may used to attach, thread, or fit a hanging feature 12 to the inverted container 1 that allows the user to hang the inverted container 1 from a support in the shower or bath. The aperture 2 in the inverted container 1 may also be located on the side or sides of the inverted container 1. The inverted container 1 may comprise more than one aperture 2.

The inverted container 1 of the present invention comprises a valve 4, which is generally positioned in a dispensing orifice 9, located in the cap portion 7, or directly on the cap portion 7 of the inverted container 1, as shown in FIG. 2. The dispensing orifice 9 is located on the bottom side of the container 1 wherein the dispensing orifice 9 can be located in the center of the bottom side, on the left or right corner of the bottom side, or slightly skewed to the right or left of the center of the bottom, such that the orifice 9 is primed with personal care composition as the container hangs from a support in the shower or bath. The valve 4 serves to regulate the dispensing of the personal care composition from the container. The valve 4 is preferably a slit valve 4 that is actuated by manually squeezing the side walls of the inverted container 1. Preferably the inverted container 1 has a slit valve 4. The slit valve 4 is an inert, generally flexible material. For example, the material is a silicone rubber or similar material.

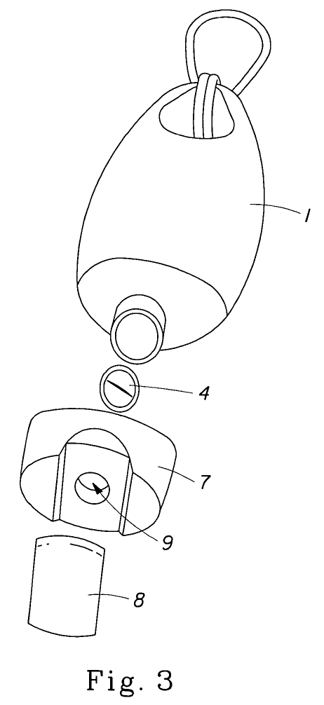

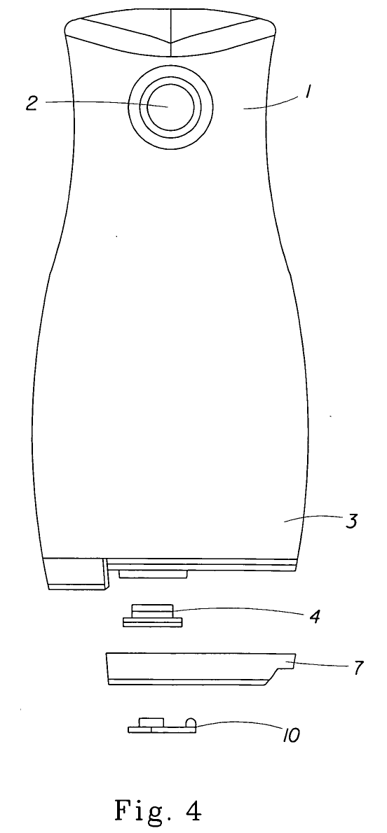

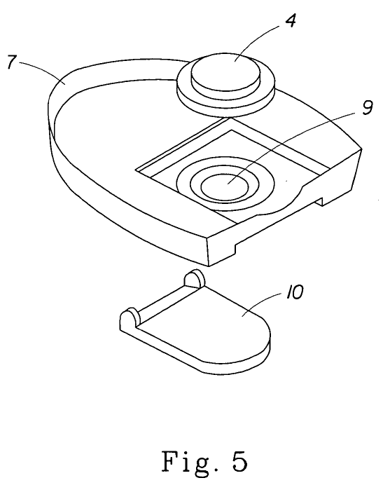

The inverted container 1 can further comprise an orifice cover which serves to cover the orifice 9 to prevent accidental dispensing of the personal care composition, e.g. during shipment of the article of commerce to retail stores. The orifice cover can be a removable cover 8, slider cover, a push/pull cover, a rotating cover, a snap cover, swing cover 10, and the cover can be hinged or non-hinged. FIG. 2 illustrates an embodiment of the slit valve 4 located in the cap portion 7 with removable orifice cover 8. FIG. 3 illustrate one possible embodiment of the article of commerce, with a slit valve 4 which is located in the cap portion 7, where the orifice 9 is located which can be covered with a removable orifice cover 8. FIG. 4 illustrates one possible embodiments of an inverted container 1 which also comprises a slit valve 4, the cap portion 7, and a swing orifice cover 10. FIG. 5 illustrates one possible embodiment of the cap portion 7 is also where the slit valve 4 is located in the cap portion 7 and orifice 9 can be covered with a swing orifice cover 10. In some embodiments where the orifice cover is a removable cover 8, the consumer can remove and discard the orifice cover, upon initial use of the article of commerce. In such embodiments, the consumer can then dispense the composition by simply grasping the inverted container 1 and squeezing the side walls of the container to actuate the valve 4 and dispense the composition from the inverted container 1. After dispensing, the consumer releases the container from her grasp, and the valve 4 will prevent additional composition from leaving the container, without having to close or replace an orifice cover.

The user dispenses the composition from the inverted container 1 and can apply a smooth layer to specific areas of the skin and/or hair. In a preferred embodiment the composition is dispensed from the inverted container 1 and applied to the entire body. In an embodiment the inverted container 1 comprises an aperture that allows the user to hang the inverted container 1 from a support in the shower or bath. In a preferred embodiment the inverted container 1 can comprise a hanging feature 12. The hanging feature 12 allows the user to hang the inverted container 1 from a support in the shower or bath, including a bar, rack, door handle, spigot or shower head.

The hanging feature 12 can be comprised of an elastomeric or stretchable material including but not limited to rubber, TPE. TPR, TPU, elastic bands, elastic cords, elastic fabrics, silicon, TPV, and mixtures thereof. The hanging feature 12 can be made of any non-stretchable material including but not limited to polypropylene, polyethylenes of all kinds, polycarbonate, acrylic, plastics, fabrics, ropes, metals, and mixtures thereof. Preferably, the hanging feature 12 will be comprised of an elastomeric or stretchable material. The user's experience is also further enhanced when the hanging feature is comprised of an elastomeric or stretchable material because the material allows the inverted container 1 can be positioned in various configurations for ease of dispensing in the shower or bath.

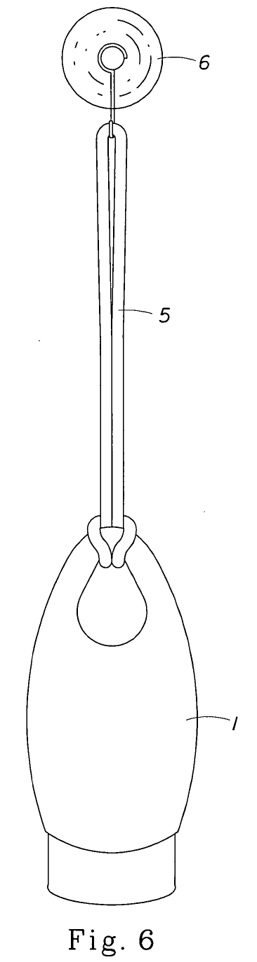

The hanging feature 12 can include the following examples a tether, a hook, a sling, a strap, and a c-hook. FIG. 6 illustrates one possible embodiment of an inverted container 1 comprising a tether 5 attached to one end of the inverted container 1. Additionally, FIG. 6 also comprises a suction cup 6 that has the tether 5 attached there from. In a preferred embodiment the inverted container 1 has a hanging feature 12 and a suction cup which allows the user to place the suction cup 6 inside the shower or bath and hang the hanging feature 12 there from. The suction cup 6 in one embodiment can be a constitutive part of the inverted container 1. The hanging feature 12 may also comprise a sleeve or a net that surrounds the inverted bottle 1. The hanging feature 12 may be transparent, translucent, opaque or mixtures or transparent, translucent, and opaque. Preferably, the hanging feature is transparent. The hanging feature 12 may be any color, including but not limited to red, orange, yellow, green blue, purple, but blue is the preferred color. The hanging feature 12 may match the color of the cap portion 7, orifice cover, or the personal care composition itself. The hanging feature 12 can be attached to one end of the container, separate from the container; removable from the container, an integral part of the container, disconnected from one area of the inverted container 1 and then reconnected in another area of the inverted container 1.

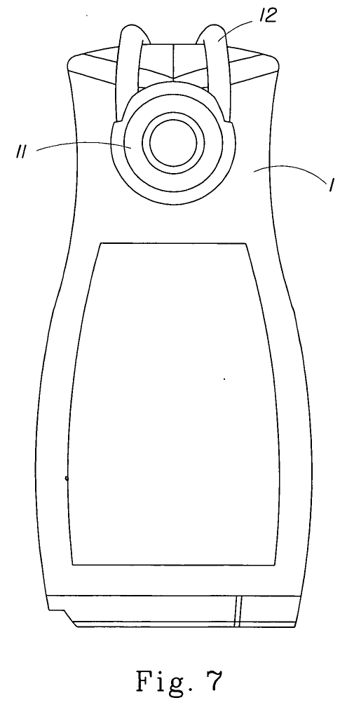

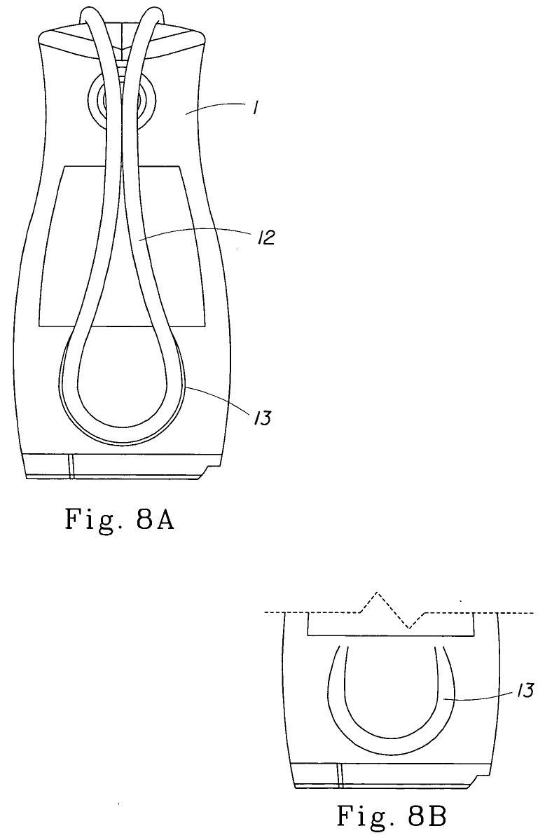

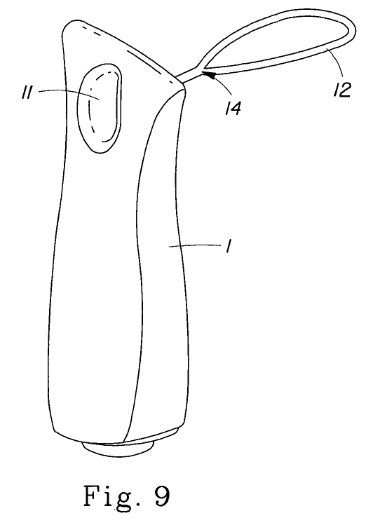

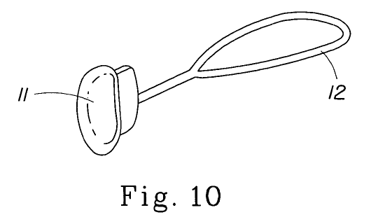

The hanging feature 12 may be fitted into the aperture 2 of the inverted container 1. Preferably, the hanging feature 12 may comprise a button 11 that may be fitted into the aperture 2 of the inverted container 1. FIG. 7 is illustrates one possible embodiment of front side of an inverted container 1 that comprises a hanging feature 12 attached to a button 11 of the inverted container 1. In one embodiment, shown in FIG. 8a and FIG. 8b the inverted container comprises a hanging feature 12 and a grooved region. The grooved region provides for an area in which the hanging feature 12 can be secured to the inverted container 1. Referring to FIG. 8a is illustrated one possible embodiment of the back side of the inverted container 1 of FIG. 7 comprising a hanging feature 12 secured to a groove region 13 of the inverted container 1. Referring to FIG. 8b is illustrated one possible embodiment of the groove region 13 of the inverted container 1 in which the hanging feature 12 of FIG. 8a can be secured. In an alternative embodiment, FIG. 9 the inverted container 1 has a hanging feature 12 and a button 11 that can be fitted into the aperture 2. The button 11 allows the user to place the inverted container 1 inside the shower or bath and hang the hanging feature 12 there from. FIG. 10 illustrates one possible embodiment of the hanging feature 12 has an attached to a button 11, which is separate from the inverted container 1. In some embodiments where the hanging feature 12 is packaged separately from the inverted container 1, the consumer can fit the hanging feature 12 through the aperture 2 and hang the inverted container 1 from a support in the shower or bath before initial use of the article of commerce.

In some embodiments, the hanging feature 12 is loop shaped. Some loop shaped embodiments may have a divider 14, as shown in FIG. 9 between the attached/or integral end and unattached end. In some embodiments, the loop may be sized such that from the unattached end to the divider 14 and from the divider 14 to the attached/or integral end would be no larger than 14 inches in diameter if the loop were to be weighted with a 5 pound weight, as described in ASTMF963-96a.

The hanging feature 12 also may comprise an area where a design, logo, brand name, trademark, coupon, rebate, or an advertisement may reside. This design, logo, brand name, trademark, coupon, rebate or advertisement may be for the personal care comprised in the inverted container 1 or may be any other personal care composition, including a personal care composition that would coordinate with the personal care composition in the inverted container 1. For, example if the personal skin care composition contained in the inverted container 1 is a shampoo, the advertisement may be for a conditioner. As well, if the personal cleansing composition in the inverted container 1 is an in-shower body moisturizer, the advertisement may be for a body wash. The area comprising the design, logo, brand name, trademark, coupon, rebate, or advertisement may be located any where on the hanging feature 12 including the button 11, the divider 14.

The efficacy of the current invention can be linked to the ability of the consumer to understand the usage instructions and to use the product accordingly. The article of commerce can further comprise a set of instructions in association with the inverted container 1 which instruct a consumer to carry out the methods of the present invention. In embodiments where the personal care composition is an in-shower body moisturizer, the set of instructions will generally comprise instructions to dispense the in-shower moisturizing composition from the inverted container 1, contact skin with the in-shower moisturizing composition during showering or bathing, rinse the skin, and then dry the skin. These instructions may comprise illustrations.

Personal Care Kit

The present invention may also comprise a personal care kit. The personal care kit of the present invention may comprise (a) an article of commerce comprising an inverted container 1 comprising a valve and an aperture 2 and a personal care composition contained in the inverted container 1; and (b) a hanging feature 12. The hanging feature 12, as described in detail above, is preferably not attached to the inverted container 1, but the hanging feature 12 is later attached by the user. This personal care kit may further comprise additional feature selected from the group consisting of cleansing puff, a suction cup 6, a suction cup 6 comprising a hook, an adhesive hook, and mixtures thereof. In some embodiments, the personal care kit may comprise (a) an article of commerce comprising an inverted container 1 comprising a valve and an aperture 2 and a personal care composition contained in the inverted container 1; (b) a hanging feature 12 and (c) suction cup 6 comprising a hook. The personal care kit may further comprise an additional personal care composition either a full size, a sample size or both. The personal care kit may further comprise an additional personal care composition that coordinates with the personal care composition that is comprised within the inverted container 1. For, example if the personal skin care composition contained in the inverted container 1 is a shampoo, the coordinating personal care composition may be for a conditioner. As well, if the personal cleansing composition in the inverted container 1 is an in-shower body moisturizer, the coordinating personal care composition may be a body wash. The personal care kit may further comprise a coupon, rebate, or advertisement. The personal care composition may further comprise a grooming device selected from the group consisting of a cleansing cloth, disposable cleansing cloth, pumice stone, a razor, brush, comb, mirror and mixtures thereof.

The personal care kit may further comprise a set of instructions. In some embodiments, the personal care composition of the personal care kit may be an in-shower moisturization composition. In embodiments where the personal care kit comprises an in-shower body moisturizer, the set of instructions would comprise instructions to dispense the in-shower moisturization composition from the inverted container 1, contact skin with the in-shower moisturization composition during showering or bathing, rinse said skin, and dry said skin. These instructions may also include illustrations.

Method of Use

The present invention also relates to a method of moisturizing skin during showering or bathing, the method comprising the steps of: (a) dispensing a in-shower moisturization composition from an inverted container 1 comprising a valve 4; (b) contacting the skin with the in-shower moisturization (c) rinsing the skin with water, d) drying the skin. The in-shower moisturization composition is preferably applied to the entire body. Preferably the valve located in the orifice of the inverted container is a slit valve 4. The inverted container further comprises a removable orifice cover 8. The method can further comprise the step of removing the removable orifice cover 8 from the inverted container prior to dispensing the composition there from. In embodiments, the set of instructions can also include the instructions to dispense multiple doses of the in-shower moisturization composition. The inverted container 1 can further comprise an aperture 2 and a hanging feature 12, wherein the hanging feature 12 allows the user to dispense the composition while hanging in the shower. The method can further comprise the step of hanging the hanging feature 12 from a support in the shower prior to dispensing the composition there from.

The present invention also relates to a method of moisturizing skin during showering or bathing, the method comprising the steps of: (a) dispensing a personal care composition from an inverted container 1 comprising a valve 4; (b) contacting the skin with the personal care composition (c) rinsing the skin with water, d) drying the skin. The personal care composition is preferably applied to the entire body. Preferably the valve located in the orifice of the inverted container is a slit valve 4. The inverted container further comprises a removable orifice cover 8. The method can further comprise the step of removing the removable orifice cover 8 from the inverted container prior to dispensing the composition there from. In embodiments, the set of instructions can also include the instructions to dispense multiple doses of the in-shower moisturization composition. The inverted container 1 can further comprise an aperture 2 and a hanging feature 12, wherein the hanging feature 12 allows the user to dispense the composition while hanging in the shower. The method can further comprise the step of hanging the hanging feature 12 from a support in the shower prior to dispensing the composition there from.

The present invention also relates to a method of washing or conditioning the hair during showering or bathing, said method comprising the steps of: (a) dispensing a personal care composition from an inverted container 1 comprising a valve 4; (b) contacting the hair with the personal care composition (c) rinsing the hair with water, d) drying the skin. Preferably the valve located in the orifice of the inverted container is a slit valve 4. The inverted container further comprises a removable orifice cover 8. The method can further comprise the step of removing the removable orifice cover 8 from the inverted container prior to dispensing the composition there from. In embodiments, the set of instructions can also include the instructions to dispense multiple doses of the in-shower moisturization composition. The inverted container 1 can further comprise an aperture 2 and a hanging feature 12, wherein the hanging feature 12 allows the user to dispense the composition while hanging in the shower. The method can further comprise the step of hanging the hanging feature 12 from a support in the shower prior to dispensing the composition there from.

Personal Care Composition

The personal care composition of the present invention may be shampoo, conditioner, a body wash, skin care treatment, moisturizer, or an in-shower moisturization composition. The personal care composition may comprise one phase or multiple phases.

The personal care compositions can comprises a skin compatible oils, emulsions, HIP emulsions, water in oil emulsions, oil in water emulsions, gel networks, non-aqueous systems, and polyerically stabilized dispersions. Preferably the in-shower moisturization compositions of the present invention are selected from the group consisting of oils, oil in water emulsions, gel networks, and mixtures thereof. Preferably the in-shower moisturization compositions are oil in water emulsions. Examples of in-shower moisturization compositions are described in detail in U.S. application No. 60/515,030 filed May 9, 2003, U.S. Publication No. 2003/0190296, U.S. Publication No. 2003/0054019, U.S. Publication No. 2003/0049282, U.S. Pat. No. 6,699,488 and U.S. Pat. No. 6,645,511.

1. Skin Compatible Oil

The personal care composition of the present invention can comprise a skin compatible oil which is defined here, as an oil that is liquid or semi-solid at the temperature at which bathing is carried out that is deemed safe for use in cosmetics being either inert to the skin or actually beneficial. The composition comprises no more than about 80 weight percent of said skin compatible oil, preferably no more than about 70 weight percent, still more preferably no more than about 60 weight percent, and most preferably no more than about 50 weight percent of the skin compatible oil. The composition comprises at least about 1 weight percent, preferably at least about 5 weight percent, even more preferably at least about 7 weight percent, and most preferably at least about 10 weight percent of the skin compatible oil. The most useful skin compatible oils for the present invention include ester oils, hydrocarbon oils, and silicone oils. Examples of these skin compatible oils are described in U.S. application No. 60/515,030 filed May 9, 2003, U.S. Publication No. 2003/0190296, U.S. Publication No. 2003/0054019, U.S. Publication No. 2003/0049282, U.S. Pat. No. 6,699,488 and U.S. Pat. No. 6,645,511.

2. Oil-In Water Emulsion

The in-shower moisturization composition of the present invention can comprise an oil in water emulsion. The oil in water emulsion comprises a skin compatible oil and an aqueous phase. In a preferred embodiment the oil in water emulsion comprises an aqueous continuous phase, a discontinuous structured oil phase, and an aqueous phase stability agent. In some embodiments, compositions are effectively ‘non-lathering’. In a preferred embodiment the composition comprises less than about 2% by weight of the composition, of an anionic surfactant, preferably less than about 1.5%, more preferably less than about 1.0%, even more preferably less than about 0.5% by weight of the composition of an anionic surfactant.

A. Aqueous Phase

When present the continuous aqueous phase generally comprises from no more than about 90 weight percent of a fluid, preferably no more than about 80 weight percent, even more preferably no more than about 70 weight percent, still more preferably no more than about 60 weight percent. The continuous aqueous phase of the present invention typically comprises at least 10 weight percent of a fluid, preferably at least 20 weight percent, even more preferably at least 30 weight percent, still more at least 40 weight percent of a fluid. The aqueous phase is the continuous phase of the instant composition in which the structured oil phase is dispersed. The aqueous phase contains the aqueous phase stability agent, and optionally such ingredients as preservatives, wetting agents, auxiliary emulsifiers and various optional benefit agents.

B. Structured Oil Phase

The structured oil phase comprises two essential components: a skin compatible oil, and a structurant that can form a stable network at a temperature below 35° C.

C. Skin Compatible Oil

A skin compatible oil is defined here, as an oil that is liquid or semi-solid at the temperature at which bathing is carried out that is deemed safe for use in cosmetics being either inert to the skin or actually beneficial. The composition comprises no more than about 80 weight percent of said skin compatible oil, preferably no more than about 70 weight percent, still more preferably no more than about 60 weight percent, and most preferably no more than about 50 weight percent of the skin compatible oil. The composition comprises at least 1 weight percent, preferably at least 5 weight percent, even more preferably at least 7 weight percent, and most preferably at least 10 weight percent of the skin compatible oil. The most useful skin compatible oils for the present invention include ester oils, hydrocarbon oils, and silicone oils. Examples of these skin compatible oils are described in U.S. application No. 60/515,030 filed May 9, 2003, U.S. Publication No. 2003/0190296, U.S. Publication No. 2003/0054019, U.S. Publication No. 2003/0049282, U.S. Pat. No. 6,699,488 and U.S. Pat. No. 6,645,511.

D. Structurant

The second component of the structured oil phase is a structurant. The structurant must satisfy two requirements. Firstly, the structurant must be capable of forming a stable network of in the skin compatible oil phase at a temperature below 35° C. This property is critical so that the structured oil is active during use but is not perceived as gritty. By stable, we mean the network survives at least one month of storage at 25° C. and 35° C.

The second requirement is that the structurant provides structured oil phase with the correct rhelogical properties. The structured oil phase should have a viscosity in the range of 100 to about 200,000 poise measured at 1 Sec-1, preferably 200 to about 100,000 poise, and most preferably 200 to about 50,000 poise as determined using the lipid rheology method described in U.S. application No. 60/515,030 filed May 9, 2003. The amount of structurant required to produce this viscosity will vary depending on the oil and the structurant, but in general, the structurant will preferably be no more than about 75 weight percent of the structured oil phase, more preferably no more than about 50 weight percent, and still more preferably no more than about 35 weight percent of the structured oil phase. Structurants meeting the above requirements with the selected skin compatible oil can form 3-dimensional network to build up the viscosity of the selected oils. It has been found that such structured oil phases, i.e., built with the 3-dimensional network, are extremely desirable for use as wet-skin treatment compositions used in bathing. These structured oils can deposit and be retained very effectively on wet skin and retained after rinsing and drying to provide long-lasting after wash skin benefit without causing a too oily/greasy wet and dry feel. It is believed that the highly desirable in-use and after-use properties of such structured oils are due to their shear thinning Theological properties and the weak structure of the network. Due to its high low-shear viscosity, the solid-network structured oil can stick and retain well on the skin during application of the skin conditioner. After being deposited on the skin, the network yields easily during rubbing due to the weak structuring of the crystal network and its lower high-shear viscosity.

The degree of shear-thinning (which is described in the Lipid Rheology Method described in U.S. application No. 60/515,030 filed May 9, 2003 exhibited by the structured oil phase is given by the value of n from the Power Law Model. Newtonian fluids which exhibit no shear thinning properties have n values close to one, while lower values indicate that the structured oil phase is more shear-thinning. For the present invention, it is preferred that the structured oil phase have a shear index less than 0.8, more preferably less than 0.6, even more preferably less than 0.5 and most preferably less than 0.4.

The structurant can be an organic structurant that is either crystalline solids or amorphous gels with molecular weight less than 5,000 Daltons, preferably less than 3,000 Daltons. Preferred organic structurants have a melting point greater than 35° C., preferably greater than 40° C. Especially preferred structurants are those that can form a solution with the selected skin compatible oil at a temperature higher than their melting point to form a free flowing clear solution. Upon cooling to the ambient temperature, the organic structurant precipitate from the oil phase to form a 3-dimensional structure providing the physical properties set forth above. Examples of organic thickeners suitable for the invention are solid fatty acid esters, natural or modified fats, fatty acid, fatty amine, fatty alcohol, natural and synthetic waxes, and petrolatum. Petrolatum is a preferred organic structuring agent. Examples of these structurants are further described in U.S. application No. 60/515,030 filed May 9, 2003, U.S. Publication No. 2003/0190296, U.S. Publication No. 2003/0054019, U.S. Publication No. 2003/0049282, U.S. Pat. No. 6,699,488 and U.S. Pat. No. 6,645,511.

E. Aqueous Phase Stability Agent

The compositions of the present invention can include one or more aqueous phase stability agent. Preferably the aqueous phase stability agent is a polymeric stabilizer. When present, the composition preferably comprises no more than about 10 weight percent, more preferably no more than 8 weight percent, and still more preferably no more than 7 weight percent, by weight of the composition of the polymeric stabilizer. When present, the composition preferably comprises at least 0.01 weight percent, more preferably at least 0.05 weight percent, and still more preferably at least 0.1 weight percent, by weight of the composition of the polymeric stabilizer. A better method of describing the polymer stabilizer is to say that it must build viscosity in the product. This can be measured using the Polymeric Stabilizer Viscosity Test as described in application number U.S. application No. 60/515,030 filed May 9, 2003. Preferably, the stability agent produces a viscosity in this test of at least 1000 cps, more preferably at least 1500 cps, and still more preferably at least 2000 cps.

Nonlimiting examples of polymeric stabilizers useful herein include carboxylic acid polymers, polyacrylamide polymers, crosslinked polyacrylate polymers, polysaccharides, gums, modified starches. Examples of these polymeric stabilizers are further described in U.S. application No. 60/515,030 filed May 9, 2003 and U.S. Pat. No. 6,699,488

3. Gel-Network

The present invention can comprise a gel network. When present the ‘gel-network’ of the present invention is composed of a hydrophobic structuring agent and a non-ionic, hydrophilic surfactant. Preferred levels of these individual components are specified below; however, the total gel-network portion of the composition is limited separately from its individual components. Without being bound by theory, it is believed that the gel-network allows for good application of the product in the wet environment of bathing. As the product is applied it is diluted with the water present on the skin and possibly that of the bath or shower. The gel-network allows for a ‘smooth’ dilution of the product, allowing the product to be spread easily and deposit evenly on the skin of the user. However, higher levels of gel-network interfere with deposition, effectively releasing fewer lipids from the composition and result in more rinse-off, or lower deposition efficiency. For this reason, when the gel net work is present the level of gel-network in the product is preferably kept relatively low. In a preferred embodiment, the gel network will not form a homogeneous aqueous phase as described in the gel-network stability test described in application number U.S. application No. 60/515,030 filed May 9, 2003. Without the gel-network in the product, i.e. a product consisting essentially of water, aqueous phase stability agent and lipid, application characteristics are undesirable and the product is difficult to spread and deposit evenly.

The present invention comprises no more than about 20 weight percent, preferably no more than about 10 weight percent, and more preferably no more than about 5 weight percent, of a hydrophobic, structuring agent selected from the group consisting of saturated C₁₆ to C₃₀ fatty alcohols, saturated C₁₆ to C₃₀ fatty alcohols containing from about 1 to about 5 moles of ethylene oxide, saturated C₁₆ to C₃₀diols, saturated C₁₆ to C₃₀ monoglycerol ethers, saturated C₁₆ to C₃₀ hydroxy fatty acids, and mixtures thereof, having a melting point of at least about 40° C. The present invention preferably comprises at least 0.5 weight percent, more preferably at least 1 weight percent, even more preferably at least 2 weight percent, and still more preferably at least 3 weight percent, of a hydrophobic, structuring agent selected from the group consisting of saturated C₁₆ to C₃₀ fatty alcohols, saturated C₁₆ to C₃₀ fatty alcohols containing from about 1 to about 5 moles of ethylene oxide, saturated C₁₆ to C₃₀diols, saturated C₁₆ to C₃₀ monoglycerol ethers, saturated C₁₆ to C₃₀ hydroxy fatty acids, and mixtures thereof, having a melting point of at least about 40° C. Without being limited by theory, it is believed that these structuring agents are useful to assist in the formation of the rheological characteristic of the composition which contribute to the hydrolytic stability of the composition of the present invention. In particular, structuring agents assist in the formation of the liquid crystalline gel network structures.

The preferred structuring agents of the present invention are selected from the group consisting of stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. More preferred structuring agents of the present invention are selected from the group consisting of stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. Even more preferred structuring agents are selected from the group consisting of stearyl alcohol, cetyl alcohol, behenyl alcohol, steareth-2, and mixtures thereof.

A. Hydrophilic Surfactant

When present the personal care compositions of the present invention can comprise no more than about 10 weight percent, preferably no more than about 6 weight percent, and more preferably no more than about 3% of at least one hydrophilic surfactant. The compositions of the present invention can comprise at least 0.1 weight percent, preferably at least 0.2 weight percent, and more preferably at least 0.3 weight percent of at least one hydrophilic surfactant. Without being limited by theory, it is believed that the hydrophilic surfactant disperses the hydrophobic materials, i.e. the structuring agent, in the water phase. The surfactant, at a minimum, must be hydrophilic enough to disperse in water.

The exact surfactant chosen will depend upon the pH of the composition and the other components present. Preferred for use herein are nonionic surfactants. Examples of these nonionic surfactants are further described in application U.S. application No. 60/515,030 filed May 9, 2003.

Emulsifier

In addition, there are several commercial emulsifier mixtures that can be useful in some embodiments. Nonlimiting examples include PROLIPID 141 (glyceryl stearate, behenyl alcohol, palmitic acid, stearic acid, lecithin, lauryl alcohol, myristyl alcohol and cetyl alcohol) and 151 (Glyceryl stearate, cetearyl alcohol, stearic acid, 1-propanamium, 3-amino-N-(2-(hydroxyethyl)-N-N-Dimethyl, N-C(16-18) Acyl Derivatives, Chlorides) from ISP; POLAWAX NF (Emulsifying wax NF), from Croda; and EMULLIUM DELTA (cetyl alcohol, glyceryl stearate, peg-75 stearate, ceteth-20 and steareth-20) from Gattefosse.

Benefit Agents

The personal care compositions of the present invention can comprise one or more benefit agents. Preferably when the in-shower moisturization composition comprises an oil in water emulsion, the benefit agent is dispersed within the skin compatible oil. When the oil in water emulsion comprises a structured oil phase the benefit agent is preferably dispersed within the structured oil phase.

In a preferred embodiment, where the composition is to be in contact with human keratinous tissue, the benefit agents should be suitable for application to keratinous tissue, that is, when incorporated into the composition they are suitable for use in contact with human keratinous tissue without undue toxicity, incompatibility, instability, allergic response, and the like within the scope of sound medical judgment.

The CTFA Cosmetic Ingredient Handbook, Second Edition (1992) describes a wide variety of nonlimiting cosmetic and pharmaceutical ingredients commonly used in the personal care industry, which are suitable for use in the compositions of the present invention.

In any embodiment of the present invention, however, the benefit agents useful herein can be categorized by the benefit they provide or by their postulated mode of action. However, it is to be understood that the additional benefit agents useful herein can in some instances provide more than one benefit or operate via more than one mode of action. Therefore, classifications herein are made for the sake of convenience and are not intended to limit the active to that particular application or applications listed.

Benefit agents are selected from the group consisting of vitamins and derivatives thereof (e.g., ascorbic acid, vitamin E, tocopheryl acetate, and the like); sunscreens; thickening agents (e.g., polyol alkoxy ester, available as CROTHIX from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol), antibacterial agents, vitamins, zeolites, peptides, terpene alcohols, desquamation actives, anti-wrinkle actives, anti-atrophy actives, flavanoids, anti-inflammatory agents, anti-cellulite agents, topical anesthetics, tanning actives, skin lightening agents, anti-fungal actives, skin moisturizing actives, cosmetic actives, petrolatum, fatty acids, esters of fatty acids, fatty alcohols, ethoxylated alcohols, polyol polyesters, glycerine, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, petrolatum, petrolatum derivatives, synthetic waxes, polyolefinic glycols, polyolefinic monoester, polyolefinic polyesters, cholesterols, cholesterol esters, interference pigments, hydrophobically modified interference pigments, shiny particles, and mixtures thereof. These materials can be used at ranges sufficient to provide the required benefit, as would be obvious to one skilled in the art. Examples of these benefit agents are further described in application U.S. application No. 60/515,030 filed May 9, 2003, U.S. application No. 60/515,029 filed May 8, 2003, U.S. Publication No. 2003/0190296, U.S. Publication No. 2003/0054019, U.S. Publication No. 2003/0049282, U.S. Pat. No. 6,699,488 and U.S. Pat. No. 6,645,511.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and claims, are by weight and all numerical limits are used with the normal degree of accuracy afforded by the art, unless otherwise specified.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

All exemplified amounts are concentrations by weight of the total in-shower moisturization compositions, unless otherwise specified.

| | Ex. 1 | Ex. 2 | Ex. 3 | Ex. 4 | Ex. 5 | Ex. 6 | Ex. 7 | Ex. 8 | Ex. 9 |
|---|---|---|---|---|---|---|---|---|---|
| Ingredient | wt % | Wt % | wt % | Wt % | wt % | wt % | wt % | wt % | wt % |
| Hydroxypropyl Starch | 3.5 | 4.0 | 3.0 | 4.0 | 3.5 | 3.5 | 4.0 | 4.0 | 3.5 |
| Phosphate (Structure XL | | | | | | | | | |
| from National Starch) | | | | | | | | | |
| Emulsifying Wax NF | 2.75 | 3.0 | 2.5 | 3.0 | 2.75 | | 3.0 | 3.0 | 1 |
| (Polawax from Croda) | | | | | | | | | |
| Tween 60 (Polysorbate-60 | | | | | | 0.5 | | | |
| from ISP) | | | | | | | | | |
| Cetyl Alcohol | | | | | | 0.4 | | | |
| Stearyl Alcohol | | | | | | 0.4 | | | |
| Fragrance | 1.0 | 1.0 | 1.0 | 1.0 | 1.0 | 1.0 | 1.0 | 1.0 | 1.0 |
| Preservatives | 0.8 | 0.8 | 0.8 | 0.8 | 0.8 | 0.8 | 0.8 | 0.8 | 0.8 |
| Water | Q.S. | Q.S. | Q.S. | Q.S. | Q.S. | Q.S. | Q.S. | Q.S. | Q.S. |
| Petrolatum (Superwhite | 15 | 15 | 30 | 25 | 20 | 20 | 15 | | 35 |
| Protopet from WITCO) | | | | | | | | | |
| Mineral Oil (Hydrobrite 1000 | | | | | 5 | | | | |
| PO White MO from WITCO) | | | | | | | | | |
| Jojoba Oil (Lipovol J from | | | | 5 | | | | | |
| Lipo) | | | | | | | | | |
| Silicone Fluid (50 cstk from | | | | | | | 2 | | |
| Dow Corning) | | | | | | | | | |
| Gelled Mineral Oil (Versagel | | | | | | | | 15 | |
| M750 from Penreco) | | | | | | | | | |

The in-shower moisturization composition of Example 1-9 can be prepared by conventional formulation and mixing techniques. One such example is shown below, although a variety of orders of addition can be used to formulate useable products.

Prepare the aqueous phase composition by first dispersing the hydroxypropyl starch phosphate in water. Add gel network phase (emulsifying wax or tween 80/cetyl alcohol/stearyl alcohol blend) and heat to 160° F. Place mixing vessel in a water bath to cool to under 100° F. Add fragrance.

Premix all lipids at 160° F. Add to the aqueous phase (<80° F.) with increased agitation. (In the case of examples with multiple lipids, the lipids can be premixed or not, depending upon the desired outcome.) Add preservatives and agitate until product is smooth.

| | Ex. | Ex. | Ex. |
|---|---|---|---|
| | 10 | 11 | 12 |
| Ingredient | wt % | Wt % | wt % |
| Sepigel 305 from Seppic | | 0.5 | |
| Hydroxypropyl Starch | 3.5 | 2.5 | 3.5 |
| Phosphate (Structure XL from | | | |
| National Starch) | | | |
| Emulsifying Wax NF (Polawax | 3.0 | 2.25 | 3.0 |
| from Croda) | | | |
| Fragrance | 1.0 | 1.0 | 1.0 |
| Preservatives | 0.8 | 0.8 | 0.8 |
| Water | Q.S. | Q.S. | Q.S. |
| Petrolatum (Superwhite | 20 | | |
| Protopet from WITCO) | | | |
| Mineral Oil (Hydrobrite 1000 | | | 5 |
| PO White MO from WITCO) | | | |
| G-2180 Petrolatum from | | 25 | 20 |
| Crompton | | | |
| Gelled Mineral Oil (Versagel | 5 | | 5 |
| M750 from Penreco) | | | |

The in-shower moisturization composition of Example 10-12 can be prepared by conventional formulation and mixing techniques. One such example is shown below, although a variety of orders of addition can be used to formulate useable products.

First, prepare the aqueous phase composition by dispersing the hydroxypropyl starch phosphate in water. Add gel network phase (emulsifying wax or tween 80/cetyl alcohol/stearyl alcohol blend) and heat to 160° F. Place mixing vessel in a water bath to cool to under 100° F. Add fragrance.

Add the lipid(s) (preheated to 160° F.) to the aqueous phase (<80° F.) with increased agitation. (In the case of examples with multiple lipids, the lipids can be premixed or not, depending upon the desired outcome.) Add preservatives and agitate until product is smooth.

Articles of Commerce and Personal Care Kits:

Examples 1-12 are placed in an inverted container 1 comprising a slit valve as shown in FIGS. 1-9.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An article of commerce comprising:
(a) an inverted container comprising a valve and an aperture;
(b) a personal care composition contained in said inverted container; and
(c) a hanging feature.

2. The article of commerce of claim 1, wherein said hanging feature is selected from the group consisting of a tether, a hook, a sling, a strap, a c-hook and combinations thereof.

3. The article of commerce of claim 1, wherein said valve is said slit valve.

4. The article of commerce of claim 1, wherein said inverted container further comprises an orifice cover.

5. The article of commerce of claim 4, wherein said orifice cover can include a removable cover, slider cover, a push/pull cover, a rotating cover, a snap cover, and a swing cover.

6. The article of commerce of claim 1, further comprises a set of instructions.

7. The article of commerce of claim 1, wherein the personal care composition is an in-shower moisturization composition.

8. The article of commerce of claim 7, wherein said set of instructions comprising instructions to dispense said in-shower moisturization composition from said inverted container, contact skin with said in-shower moisturization composition during showering or bathing, rinse said skin, and dry said skin.

9. The article of commerce of claim 1, wherein said aperture is adjacent to the top portion of said container and allows for hanging of said inverted container from a support in the shower or bath.

10. The article of commerce of claim 1, further comprising a suction cup; wherein said suction cup is placed inside the shower or bath and allows the user to hang the hanging feature there from.

11. The article of commerce of claim 1, wherein the hanging feature is comprised of an elastomeric material.

12. A personal care kit comprising:
(a) an article of commerce comprising an inverted container comprising a valve and an aperture and a personal care composition contained in the inverted container; and
(b) a hanging feature.

13. The personal care kit of claim 12, wherein said hanging feature is selected from the group consisting of a tether, a hook, a sling, a strap, a c-hook and combinations thereof.

14. The personal care kit of claim 12 further comprising an comprise additional feature selected from the group consisting of a cleansing puff, a suction cup, a suction cup comprising a hook; an adhesive hook; and mixtures thereof.

15. The personal care kit of claim 12, wherein said valve is said slit valve.

16. The personal care kit of claim 12 wherein said hanging feature is comprised of an elastomeric material.

17. The personal care kit of claim 12 further comprising a set of instructions.

18. An article of commerce comprising:
(a) an inverted container comprising a valve
(b) an in-shower moisturization composition contained in said inverted container; and
(c) a set of instructions in association with said inverted container, said set of instructions comprising instructions to dispense said in-shower moisturization composition from said inverted container, contact skin with said in-shower moisturization composition during showering or bathing, rinse said skin, and dry said skin.

19. The article of commerce of claim 18, wherein said instructions comprise illustrations.

20. The article of commerce of claim 18, wherein said valve is said slit valve.

21. The article of commerce of claim 20, wherein said valve is located in an orifice of said inverted container.

22. The article of commerce of claim 18, wherein said inverted container further comprises an orifice cover.

23. The article of commerce of claim 22, wherein said orifice cover can include a removable cover, slider cover, a push/pull cover, a rotating cover, a snap cover, and a swing cover.

24. The article of commerce of claim 18, wherein said set of instructions further comprises an instruction to dispense multiple doses of said in-shower moisturization composition.

25. The article of commerce of claim 18, wherein said inverted container further comprises an aperture adjacent to the top portion of said container and allows for hanging of said inverted container from a support in the shower or bath.

26. The article of commerce of claim 18, wherein said inverted container further comprises a hanging feature that allows for hanging of said inverted container from a support in the shower or bath.

27. The article of commerce of claim 26, wherein said hanging feature is selected from the group consisting of a tether, a hook, a sling, a strap, a c-hook and combinations thereof.

28. The article of commerce of claim 18, further comprising a suction cup; wherein said suction cup is placed inside the shower or bath and allows the user to hang the hanging feature there from.

29. The article of commerce of claim 18, wherein said in-shower moisturization composition is selected from the group consisting of an oil in water emulsion, a skin compatible oil, a water in oil emulsion, a gel network, and mixtures thereof.

30. The article of commerce of claim 29, wherein said in-shower moisturization composition is said oil in water emulsion.

31. A method of moisturizing skin during showering or bathing, said method comprising the steps of:
a) dispensing a in-shower moisturization composition from an inverted container comprising a valve;
(b) contacting said skin with said in-shower moisturization composition;
(c) rinsing said skin with water; and
(d) drying said skin.

32. The method of claim 31, wherein said method comprises contacting said skin with multiple doses of said personal care composition.

33. The method of claim 31 wherein said valve is a slit valve.
